(19) United States
(12) Patent Application Publication
(10) Pub. No.: US 2018/0015164 A1
(43) Pub. Date: Jan. 18, 2018

(54) A Composition Containing Protective Agent for Singlet Oxygen and A Preparation Method therefor

(71) Applicant: Nanjing University, Nanjing (CN)

(72) Inventors: Yiqiao HU, Nnjing (CN); Jinhui WU, Nanjing (CN); Yuhao CHENG, Nanjing (CN); Qing HUANG, Nanjing (CN)

(73) Assignee: Nanjing University, Nanjing (CN)

(21) Appl. No.: 15/547,521

(22) PCT Filed: Dec. 29, 2016
(86) PCT No.: PCT/CN2016/113190

§ 371 Date: Jul. 31, 2017

(30) Foreign Application Priority Data
Jan. 16, 2016 (CN) 201610028413.X

(51) Int. Cl. A61K 41/00; A61K 47/44; A61K 47/42; A61K 47/18; A61K 9/107; A61K 47/06; A61K 9/16; A61K 9/127; B01J 13/08; A61K 47/34

(52) U.S. Cl. CPC A61K 41/0057; B01J 13/08; A61K 47/44; A61K 47/42; A61K 47/34; A61K 9/107; A61K 47/06; A61K 9/1682; A61K 9/1277; A61K 47/18

(57) ABSTRACT

The Invention relates to a method to utilize the characteristic of a protective agent to incubate singlet oxygen and prolong its lifetime so as to provide abundant singlet oxygen with persistence and thus improve the effect of photodynamic therapy. The composition is composed of a functional substance that can prolong the lifetime of singlet oxygen, an emulsifier and a photosensitizer; the functional substance is emulsified into an emulsion through the emulsifier and delivered into a tumor tissue with the photosensitizer; then the photodynamic therapy can be conducted with the abundant singlet oxygen incubated by the functional substance that can prolong the lifetime of singlet oxygen and thus the effect of photodynamic therapy is improved.

### TECHNICAL FIELD

The Invention is intended to prolong the lifetime of singlet oxygen substantially with a protective agent for singlet oxygen, which may improve the effect of photodynamic therapy. The Invention is attached to the field of improving the effect of photodynamic therapy and the clinical application of the same. Specifically, the Invention utilizes the ability of protective agent to prolong the lifetime of singlet oxygen and prepare nanoparticles or micron particles with the protective agent, photosensitizer and emulsifier through emulsification so as to improve the effect of photodynamic therapy.

### BACKGROUND ART

Photodynamic therapy is an antineoplastic protocol with space-time selectivity. It delivers photosensitizer into a tumor tissue for irradiating the tumor location with visible/near infrared lasers; the photosensitizer will jump to exciting state after absorbing photons; the photosensitizer in the exciting state may deliver its energy to the oxygen in ground state (triplet oxygen) to stimulate it into the exciting state (singlet oxygen). With high oxidability and reactivity, singlet oxygen may oxidize the nucleic acids, proteins and lipids in tumor tissues in a short time and cause tumor necrosis and apoptosis, but it has a short lifetime (0.1-20 μs). Therefore, the effect of photodynamic therapy is closely related to the lifetime of the singlet oxygen in tumor tissue, i.e., the longer the lifetime of the singlet oxygen in tumor tissues is, the better the therapeutic effect of photodynamic therapy will be.

The lifetime of singlet oxygen in solution is less relevant to the types of photosensitizer, but it mostly depends on the type of solution. Protective agent, which may prolong the lifetime of singlet oxygen, is generally not dissolving photosensitizer but not limited to the inert material insoluble in water, and has the ability to incubate singlet oxygen and prolong its lifetime. Then, the curing effect can be improved as the lifetime of singlet oxygen is prolonged effectively.

### SUMMARY OF THE INVENTION

The Invention is to provide a composition containing photosensitizer, emulsifier and protective agent and a preparation method therefor; the protective agent can prolong the lifetime of singlet oxygen and improve the effect of photodynamic therapy, which may solve the present problem that the effect of photodynamic therapy is limited by the short lifetime of singlet oxygen generated by photosensitizer molecule in tumor medium.

The technical scheme to realize the purpose of the Invention is as follows:

A preparation method for a composition containing protective agent for singlet oxygen, comprising the following steps:

(a) Dissolving the photosensitizer and the emulsifier with a solvent to obtain a mixed solution;

(b) Adding appropriate amount of protective agent for singlet oxygen to the obtained mixed solution and emulsifying the protective agent in ice bath with appropriate method to form nanoparticles or micron particles.

Specifically, the solvent in Step (a) is one or more of dichloromethane, trichloromethane, ethyl alcohol, methyl alcohol, propyl alcohol or a compound thereof.

Specifically, the emulsifying method in Step (b) is an extrution method, an ultrasonic method or a high-speed dispersion method.

The average size of the liposome nanoparticle according to the technical scheme thereof is 20 nm-2000 nm; the preferred one is 35-800 nm and the most preferred one is 50-300 nm.

The volume ratio of the protective agent in the liposome nanoparticle according to the technical scheme thereof is 1%-35%.

The photosensitizer in Step (a) according to the technical scheme thereof is characterized in that the photosensitizer is totally safe and non-toxic substances that can be activated by light to produce photochemical reactions; these photosensitizers can be hydrophilic, lipophilic or amphipathic. The photosensitizer is selected from porphyrin and its derivatives as ICG, Ce6 and 5-ALA; chlorophyll and its derivatives as phaeophytin, chlorin and purpurin 18; anthraquinone and its derivatives; phthalocyanin and its derivatives as zinc phthalocyanine and aluminum phthalocyanine; endogenous photosensitizer as 5-aminolevulinic acid; phycobiliproteins as phycoerythrin and phycocyanin; pentaazadentate derivatives as lutecium III pentaazadentate, quinonyl compounds, rose-bengal and fullerene; polyacetylene as benzene phenylheptatriyne; thiophenic compound as α thiophene; inorganic photosensitizers as titanium oxide (TiO₂) and zinc oxide; or photosensitizers of Chinese herbal medicines as HyPocrellin derivatives, psoralen, curcumin, hypericin, pseudohypericin, rheum emodin, riboflavin and aloe-emodin; and heptamethine cyanines as IR780 and IR775. These photosensitizers or near infrared dyes are applicable to the Invention.

Wherein, the preferred photosensitizers are IR780, IR775 and phthalocyanin, etc.

The emulsifier in Step (a) according to the technical scheme thereof is of lipids as DSPE-PEG2000, lecithin, cholesterol, DSPC, DPPC and DSPE, etc.; proteins as human albumin, hemoglobin, transferrin, immune globulin, insulin, endostatin, myohemoglobin, fibronectin, collagen, gelatin, synthetic peptide and protein, or a combination thereof; macromolecules as PVA, poloxamer, Tween, Span, Brij, Myrj, polyoxyethylene and castor oil, etc.

Wherein, the preferred carrier emulsifier is phospholipid, DSPE-PEG2000 and albumin.

In addition, in order to enable the composition of the Invention to target the specific tumor tissue or lesion location such as liver tumor, kidney tumor, bone tumor, breast cancer and uterine fibroid; the substances having high affinity to the specific tumor tissue or lesion location also can be added into the composition, such as the targeted substances formed by identifying the antibody, peptide, ligand and aptamer of the tumor; in order to enable the efficient photosensitizer to penetrate the biological membrane, a substance with the function to penetrate the biological membrane can be added to modify the photosensitizer and form various compositions with such function. The substances function to penetrate the biological membrane are derived from (but not limited to) influenza virus, VSV, SFV, sendai virus and HIV virus, or selected from synthetic cell-penetrating peptides.

In the Invention, the composition containing the protective agent that can prolong the lifetime of singlet oxygen, the photosensitizer and the emulsifier can be the composition mixed with the protective agent, the emulsifier and the photoactive substances, or the composition constituted by the protective agent that can prolong the lifetime of singlet oxygen, the photosensitizer and the emulsifier through chemical or physical methods, which can be microvesicle, micro-capsule, particulate, micro emulsion, nanoparticle and nanoemulsion. Photoactive substances wrap or adhere to the inside or surface of the microvesicle, micro-capsule, particulate, micro emulsion, nanoparticle and nanoemulsion. The microvesicle, micro-capsule, particulate, micro emulsion, nanoparticle and nanoemulsion can be (but not limited to) either existing products in direct market or home-made; the membrane materials can be lipid, polymer, albumin and polysaccharide. The core material uses one or more of the gas, the liquid and the nanoscale solid of biocompatibility with oxygen carrying capacity.

The technical scheme is further described as follows:

In addition to the above technical scheme, the Invention further provides a method to utilize the ability of protective agent to prolong the lifetime of singlet oxygen to improve the effect of photodynamic therapy; the method comprises the following steps as: (a) dissolving the photosensitizer and the emulsifier with a solvent under the conditions of 10-35° C. and pH3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent; (b) placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask; (c) adding the solvent into the round-bottom flask for ultrasonic hydration for 10-15 min so that the emulsifier can fall off and disperse into the water to form a micellar or vesicle structure, making the lipid film fall off from bottle wall and evenly disperse into the solvent; (d) adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet. Then, nano or micro emulsions with uniform size can be prepared by controlling the factors as emulsification time, output power and rotation speed. Such nano emulsions carry protective agent and photosensitizer at the same time, and can be accumulated onto the tumor location through EPR effect after intravenous injection. During photodynamic therapy, the protective agent can further prolong the lifetime of the singlet oxygen and thus brings about photodynamic therapy of better effect.

The average size of the nanoparticles or micron particles formed in the technical scheme thereof is 20 nm-2000 nm; the volume ratio of the protective agent is 1%-35%.

The solvent in Step (a) of the technical scheme thereof comprises, but not limited to, dichloromethane, trichloromethane, ethyl alcohol, methyl alcohol, propyl alcohol and a compound thereof. The method in Step (b) of the technical scheme thereof to remove the solvent comprises but not limited to spray drying, drying in water bath, drying under reduced pressure and drying under reduced pressure in water bath.

The solution in Step (c) of the technical scheme thereof comprises but not limited to water, normal saline, acetate, physiological glucose, phosphate buffer and TRIS buffer. The method in Step (c) of the technical scheme thereof to dissolve the thin film on the flask wall formed by photosensitizer and emulsifier comprises, but not limited to, ultrasonic hydration method, vortex oscillation method and water washing method. The protective agent in Step (d) of the technical scheme thereof comprises but not limited to paraffin, lipiodol, soybean oil, dichloromethane, chloroform, perfluorinated compounds and deuteroxide; wherein, the perfluorinated compounds comprises but not limited to perfluorinated alkanes, perfluorinated amines, perfluorinated crown ethers and bromo-perfluorinated alkane. Wherein, the preferred perfluorinated compounds are perfluorohexane and perfluorotributylamine. Wherein, the lifetime of singlet oxygen in various solutions according to the literatures is 2 μs in water; 20 μs in deuteroxide; 7 μs in methyl alcohol; 12 μs in ethyl alcohol; 17 μs in hexane; 60±15 μs in chloroform; 600±200 μs in perfluorohexane; 200±60 μs in carbon disulfide and 1000±200 μs in Freon 11. The emulsifying method of the protective agent in Step (d) comprises but not limited to an extrution method, an ultrasonic method or a high-speed dispersion method. Wherein, the preferred ones are ultrasonic method and high-speed dispersion method.

The other purpose of the Invention is to provide a composition prepared with the method, which can be used to prolong the lifetime of singlet oxygen and improve the effect of photodynamic therapy.

A person skilled in the art is able to understand that the scope and spirit of the Invention is variable. Meanwhile, the organic solution that may dissolve the photosensitizer and the emulsifier is various; many types of photosensitizer and emulsifier can be used; many types of protective agent can prolong the lifetime of singlet oxygen and many operation methods are feasible. The Invention will be described more specifically and clearly in the following embodiments.

The beneficial effects of the Invention lie in that:

Firstly, the volume ratio of the protective agent in the nano or micron particles formed through the method provided by the Invention can be up to 35%, which forms a method of high efficiency and low consumption; secondly, the protective agent may not only prolong the lifetime of singlet oxygen effectively, but also improve the yield of singlet oxygen. Based on the advantages, the effect of photodynamic therapy can be largely improved with less dosing.

### BRIEF DESCRIPTION OF THE DRAWINGS

FIG. 1 is a particle size distribution diagram of the liposome-perfluorohexane-IR780 nanoparticle of the Invention (with a volume ratio of perfluorohexane as 30%).

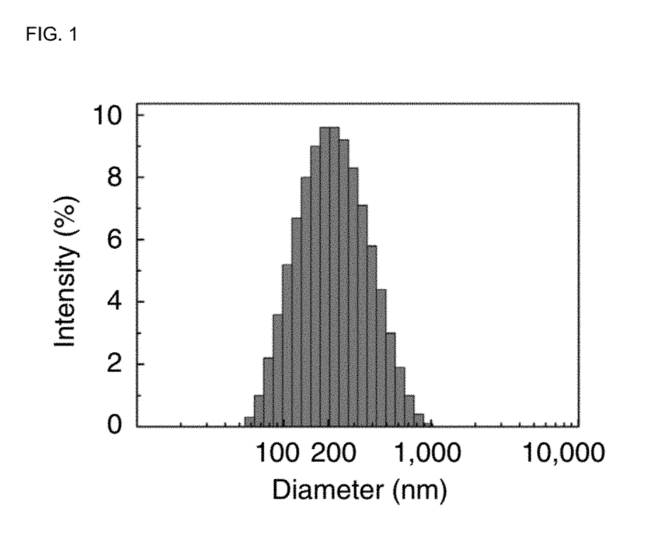

FIG. 2 is a particle size distribution diagram of the albumin-perfluorotributylamine-IR780 nanoparticle of the Invention (with a volume ratio of perfluorotributylamine as 30%).

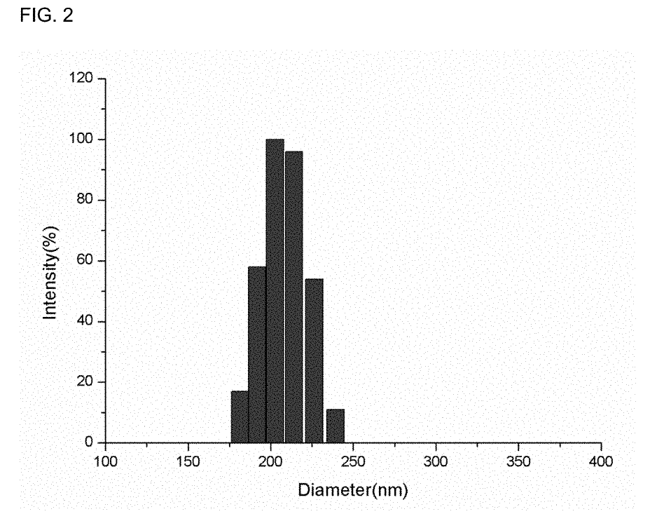

FIG. 3 is a line chart of the singlet oxygen produced by the liposome-perfluorohexane-IR780 nanoparticle of the Invention and other samples of different groups under continuous irradiation of near-infrared light.

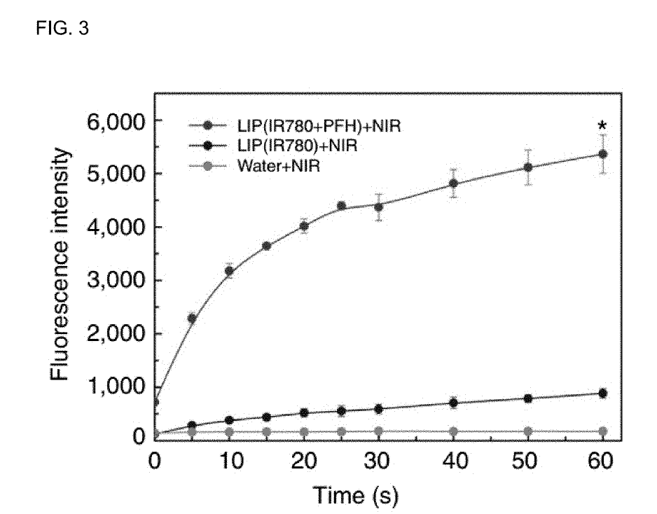

FIG. 4 is a line chart of the singlet oxygen produced by the albumin-perfluorotributylamine-IR780 nanoparticle of the Invention and other samples of different groups under continuous irradiation of near-infrared light.

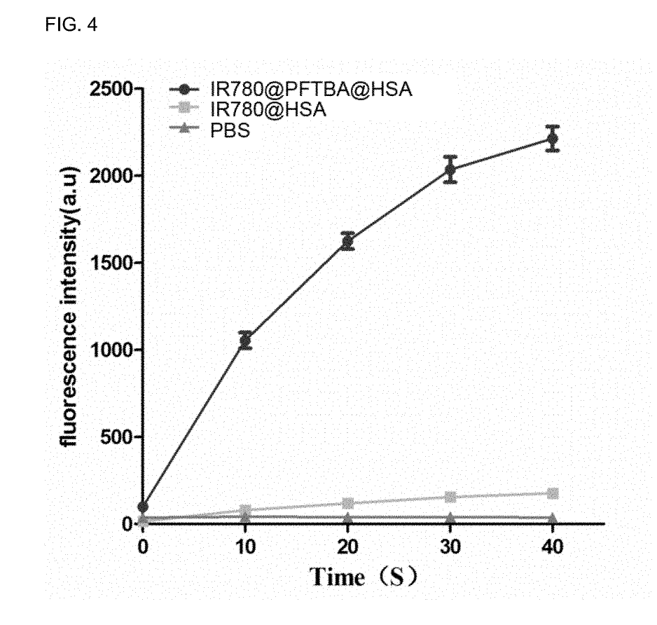

FIG. 5 is a histogram of the singlet oxygen produced by the liposome-paraffin-IR780 nanoparticle of the Invention and other samples of different groups under irradiation of near-infrared light with similar gradient dilution.

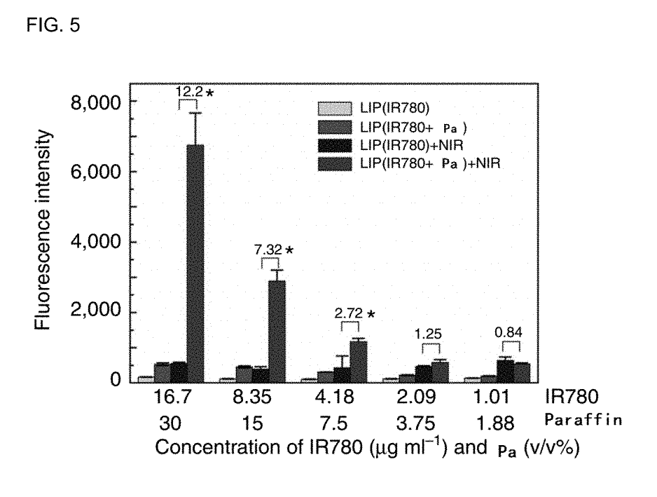

FIG. 6 is a line chart of the singlet oxygen produced by the liposome-lipiodol-IR780 nanoparticle and the liposome-IR780 nanoparticle of the Invention under continuous irradiation of near-infrared light under hypoxic conditions.

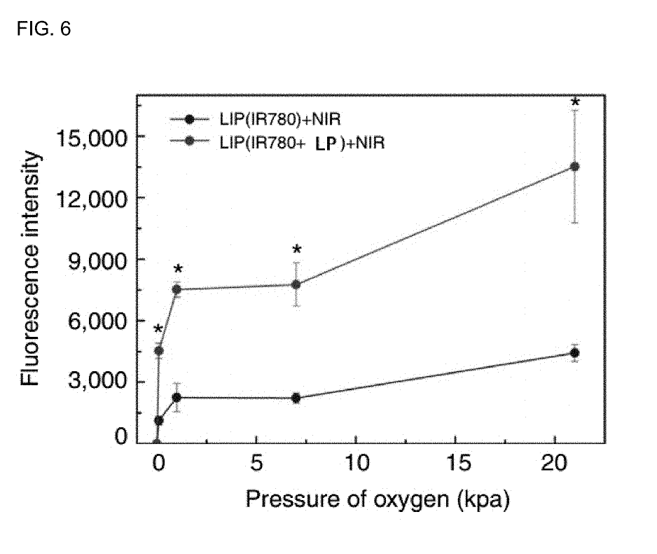

FIG. 6 is an ultraviolet absorption diagram of the liposome-perfluorohexane-IR780 nanoparticle, the liposome-IR780 nanoparticle and the IR780 solution of the Invention.

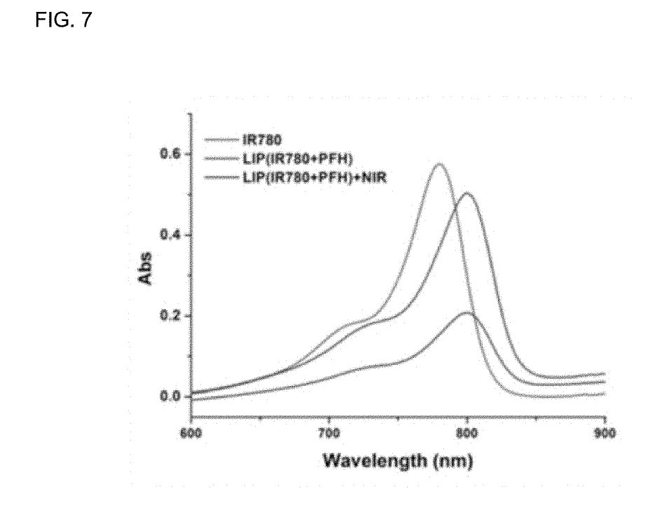

FIG. 8 is a line chart of the singlet oxygen produced by the liposome-perfluorohexane-IR780 and the IR780 solution of the Invention of different concentrations.

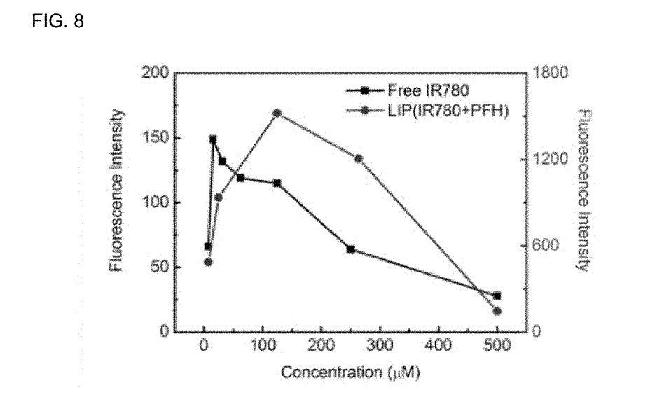

### DETAILED DESCRIPTION OF THE PREFERRED EMBODIMENTS

The followings are typical embodiments of the Invention, rather than a limitation to the scope of protection of the Invention.

### Embodiment 1. Preparation of 30 v/v% Liposome-Perfluorohexane-IR780 Nanoparticle, Containing 50 ug/ml IR780

Under the condition of pH6 and 24° C., add 24.65 mg of lecithin, 4.28 mg of cholesterol, 3.79 mg of DSPE-PEG2000 and 100 ug of IR780 into a round-bottom flask of 25 ml for being dissolved by 5 ml of dichloromethane. Then remove the dichloromethane through rotary decompression evaporation to form a lipid film carrying IR780 on the flask wall. Add 1.4 ml of normal saline into the flask for ultrasonic hydration for 10 min, making the lipid film fall off from the flask wall and evenly disperse into the normal saline. Disperse the solution at a high speed with a high-speed disperser in ice bath. Add 0.6 ml of perfluorohexane in six times (0.1 ml for each time) for 2 min of high-speed dispersion each time. After the 0.6 ml of perfluorohexane is added, keep dispersing the solution at a high speed in ice bath for 10-15 min until the particle size is uniform and the solution is stable. Then a suspension that is bright and transparent to light is obtained, consisting of the particles carrying photosensitizer with the average particle size of 50-2000 nm (analyzed with the BIC90 plus Particle Size Analyzer).

### Embodiment 2. Preparation of 30 v/v% Liposome-Paraffin-IR780 Nanoparticle, Containing 50 ug/ml IR780

Add 24.65 mg of lecithin, 4.28 mg of cholesterol, 3.79 mg of DSPE-PEG2000 and 100 ug of IR780 into a round-bottom flask of 25 ml for being dissolved by 5 ml of dichloromethane. Then remove the dichloromethane through rotary decompression evaporation to form a lipid film carrying IR775 on the flask wall. Add 1.4 ml of normal saline into the flask for ultrasonic hydration for 10 min, keeping the lipid film away from the flask wall and evenly disperse into the normal saline. Disperse the solution at a high speed with a high-speed disperser in ice bath. Add 0.6 ml of paraffin in six times (0.1 ml for each time) for 2 min of high-speed dispersion each time. After the 0.6 ml of paraffin is added, keep dispersing the solution at a high speed in ice bath for 8-10 min until the particle size is uniform and the solution is stable. Then a suspension that is bright and transparent to light is obtained, containing the particles carrying photosensitizer with the average particle size of 200-2000 nm (analyzed with the BIC90 plus Particle Size Analyzer).

### Embodiment 3. Preparation of 30 v/v% Liposome-Perfluorotributylamine-IR780 Nanoparticle, Containing 50 ug/ml IR780

Add 24.65 mg of lecithin, 4.28 mg of cholesterol, 3.79 mg of DSPE-PEG2000 and 100 ug of IR780 into a round-bottom flask of 25 ml for being dissolved by 5 ml of dichloromethane. Then remove the dichloromethane through rotary decompression evaporation to form a lipid film carrying IR780 on the flask wall. Add 1.4 ml of normal saline into the flask for ultrasonic hydration for 10 min, keeping the lipid film away from the flask wall and evenly disperse into the normal saline. Disperse the solution at a high speed with a high-speed disperser in ice bath. Add 0.6 ml of perfluorotributylamine in six times (0.1 ml for each time) for 2 min of high-speed dispersion each time. After the 0.6 ml of perfluorotributylamine is added, keep dispersing the solution at a high speed in ice bath for 10-15 min until the particle size is uniform and the solution is stable. Then a suspension that is bright and transparent to light is obtained, containing the particles carrying photosensitizer with the average particle size of 300-1200 nm (analyzed with the BIC90plus Particle Size Analyzer).

### Embodiment 4. Preparation of 30 v/v% Liposome-Lipiodol-IR780 Nanoparticle, Containing 50 ug/ml IR780

Add 24.65 mg of lecithin, 4.28 mg of cholesterol, 3.79 mg of DSPE-PEG2000 and 100 ug of IR780 into a round-bottom flask of 25 ml for being dissolved by 5 ml of dichloromethane. Then remove the dichloromethane through rotary decompression evaporation to form a lipid film carrying IR780 on the flask wall. Add 1.4 ml of normal saline into the flask for ultrasonic hydration for 10 min, making the lipid film fall off from the flask wall and evenly disperse into the normal saline. Disperse the solution at a high speed with a high-speed disperser in ice bath. Add 0.6 ml of lipiodol in six times (0.1 ml for each time) for 2 min of high-speed dispersion each time. After the 0.6 ml of lipiodol is added, keep dispersing the solution at a high speed in ice bath for 10-15 min until the particle size is uniform and the solution is stable. Then a suspension that is bright and transparent to light is obtained, containing the particles carrying photosensitizer with the average particle size of 300-1200 nm (analyzed with the BIC90 plus Particle Size Analyzer).

### Embodiment 5. Preparation of 30 v/v% albumin-soybean oil-IR780 nanoparticle, containing 50 ug/ml IR780

Add 1.4 ml of human albumin aqueous solution of 20 mg/ml and 100 ug of IR780 in an EP tube of 3 ml for mixing for 30 min at room temperature with a vortex mixer. Ultrasonic emulsify the solution in ice bath at 300 W. Add 0.6 ml of soybean oil in six times (0.1 ml for each time) for 1 min of ultrasonic emulsification each time. After the 0.6 ml of soybean oil is added, keep ultrasonic emulsification in ice bath for 2-5 min until the particle size is uniform and the solution is stable. Then a suspension that is bright and transparent to light is obtained, containing the particles carrying photosensitizer with the average particle size of 170-250 nm (analyzed with the BIC90 plus Particle Size Analyzer).

### Embodiment 6. Preparation of 30 v/v% Albumin-Perfluorotributylamine-IR775 Nanoparticle, Containing 50 ug/ml IR780

Add 1.4 ml of human albumin aqueous solution of 20 mg/ml and 100 ug of IR775 in an EP tube of 3 ml for mixing for 30 min at room temperature with a vortex mixer. Ultrasonic emulsify the solution in ice bath at 300 W. Add 0.6 ml of perfluorotributylamine in six times (0.1 ml for each time) for 1 min of ultrasonic emulsification each time. After the 0.6 ml of perfluorotributylamine is added, keep ultrasonic emulsification in ice bath for 2-5 min until the particle size is uniform and the solution is stable. Then a suspension that is bright and transparent to light is obtained, containing the particles carrying photosensitizer with the average particle size of 30-300 nm (analyzed with the BIC90 plus Particle Size Analyzer).

### Embodiment 7. Preparation of 30 v/v% Albumin-Deuteroxide-IR780 Nanoparticle, Containing 50 ug/ml IR780

Add 1.4 ml of human albumin aqueous solution of 20 mg/ml and 100 ug of IR780 in an EP tube of 3 ml for mixing for 30 min at room temperature with a vortex mixer. Ultrasonic emulsify the solution in ice bath at 300 W. Add 0.6 ml of deuteroxide in six times (0.1 ml for each time) for 1 min of high-speed dispersion each time. After the 0.6 ml of deuteroxide is added, keep ultrasonic emulsification in ice bath for 2-5 min until the particle size is uniform and the solution is stable. Then a suspension that is bright and transparent to light is obtained, containing the particles carrying photosensitizer with the average particle size of 300-1500 nm (analyzed with the BIC90 plus Particle Size Analyzer).

### Embodiment 8. Preparation of 30 v/v% Liposome-Chloroform-IR780 Nanoparticle, Containing 50 ug/ml IR780

Add 24.65 mg of lecithin, 4.28 mg of cholesterol, 3.79 mg of DSPE-PEG2000 and 100 ug of IR780 into a round-bottom flask of 25 ml for being dissolved by 5 ml of dichloromethane. Then remove the dichloromethane through rotary decompression evaporation to form a lipid film carrying IR780 on the flask wall. Add 1.4 ml of normal saline into the flask for ultrasonic hydration for 10 min, keeping the lipid film away from the flask wall and evenly disperse into the normal saline. Disperse the solution at a high speed with a high-speed disperser in ice bath. Add 0.6 ml of chloroform in six times (0.1 ml for each time) for 2 min of high-speed dispersion each time. After the 0.6 ml of chloroform is added, keep dispersing the solution at a high speed in ice bath for 10-15 min until the particle size is uniform and the solution is stable. Then a suspension that is bright and transparent to light is obtained, containing the particles carrying photosensitizer with the average particle size of 100-2000 nm (analyzed with the BIC90 plus Particle Size Analyzer).

### Embodiment 9. Preparation of 30 v/v% Liposome-Chloroform-IR780 Nanoparticle, Containing 50 ug/ml IR780

Add 24.65 mg of lecithin, 4.28 mg of cholesterol, 3.79 mg of DSPE-PEG2000 and 100 ug of IR780 into a round-bottom flask of 25 ml for being dissolved by 5 ml of dichloromethane. Then remove the dichloromethane through rotary decompression evaporation to form a lipid film carrying IR780 on the flask wall. Add 1.4 ml of normal saline into the flask for ultrasonic hydration for 10 min, keep the lipid film away from the flask wall and evenly disperse into the normal saline. Disperse the solution at a high speed with a high-speed disperser in ice bath. Add 0.6 ml of chloroform in six times (0.1 ml for each time) for 2 min of high-speed dispersion each time. After the 0.6 ml of chloroform is added, keep dispersing the solution at a high speed in ice bath for 10-15 min until the particle size is uniform and the solution is stable. Then a suspension that is bright and transparent to light is obtained, containing the particles carrying photosensitizer with the average particle size of 550-5000 nm (analyzed with the BIC90 plus Particle Size Analyzer).

### Embodiment 10. Preparation of 20 v/v% Poloxamer-Perfluorohexane-Zinc Phthalocyanine Nanoparticle, Containing 50 ug/ml IR780

Add 35 mg of poloxamer and 100 ug of IR780 into a round-bottom flask of 25 ml for being dissolved by 5 ml of trichloromethane. Then remove the trichloromethane through rotary decompression evaporation to form a lipid film carrying IR780 on the flask wall. Add 1.6 ml of normal saline into the flask for ultrasonic hydration for 10 min, making the lipid film leave from the flask wall and evenly disperse into the normal saline. Disperse the solution at a high speed with a high-speed disperser in ice bath. Add 0.4 ml of perfluorohexane in four times (0.1 ml for each time) for 2 min of high-speed dispersion each time. After the 0.4 ml of perfluorohexane is added, keep dispersing the solution at a high speed in ice bath for 3-5 min until the particle size is uniform and the solution is stable. The particles carrying photosensitizer have an average particle size of 150-1000 nm (analyzed with the BIC90 plus Particle Size Analyzer).

### Embodiment 11. Preparation of 20 v/v% Tween-Perfluorohexane-Hypericin Nanoparticle, Containing 50 ug/ml IR780

Add 47 mg of Tween and 100 ug of hypericin into a round-bottom flask of 25 ml for being dissolved by 5 ml of trichloromethane. Then remove the dichloromethane through rotary decompression evaporation to form a lipid film carrying IR780 on the flask wall. Add 1.6 ml of normal saline into the flask for ultrasonic hydration for 10 min, making the lipid film leave from the flask wall and evenly disperse into the normal saline. Disperse the solution at a high speed with a high-speed disperser in ice bath. Add 0.4 ml of perfluorohexane in four times (0.1 ml for each time) for 2 min of high-speed dispersion each time. After the 0.4 ml of perfluorohexane is added, keep dispersing the solution at a high speed in ice bath for 10-15 min until the particle size is uniform and the solution is stable. The particles with photosensitizer have an average particle size of 550-5000 nm (analyzed with the BIC90 plus Particle Size Analyzer).

The additional experiments show that liposome, protein and macromolecular can be used as an emulsifier; the obtained particle size is smaller when liposome and albumin are used as the carrier emulsifier.

During the preparation, the impact of different buffer (water, normal saline, physiological glucose, phosphate buffer, acetic acid buffer and TRIS buffer) on the particle size is observed and the result shows that normal saline is preferred.

1. A preparation method for a composition containing protective agent for singlet oxygen; the composition is composed of an emulsifier, a photosensitizer and a protective agent, wherein, the preparation method comprises the following steps:
(a) Dissolving the photosensitizer and the emulsifier with a solvent to obtain a mixed solution;
(b) Adding the protective agent for singlet oxygen into the mixed solution and emulsifying the protective agent in ice bath to prepare the composition.

2. The preparation method according to claim 1, wherein, the solvent in Step (a) is one or more of dichloromethane, trichloromethane, ethyl alcohol, methyl alcohol and propyl alcohol.

3. The preparation method according to claim 1, wherein, the emulsifying method in Step (b) is an extrution method, an ultrasonic method or a high-speed dispersion method.

4. The preparation method according to claim 1, wherein, the photosensitizer in Step (a) is hydrophilic, lipophilic or amphipathic, which is selected from one or more of porphyrin and its derivatives as ICG, Ce6 and 5-ALA; chlorophyll and its derivatives as phaeophytin, chlorin and purpurin 18; anthraquinone and its derivatives; phthalocyanin and its derivatives as zinc phthalocyanine and aluminum phthalocyanine; endogenous photosensitizer as 5-aminolevulinic acid; phycobiliproteins as phycoerythrin and phycocyanin; pentaazadentate derivatives as lutecium III pentaazadentate, quinonyl compounds, rose-bengal and fullerene; polyacetylene as benzene phenylheptatriyne; thiophenic compound as a thiophene; inorganic photosensitizers as titanium oxide (TiO₂) and zinc oxide; or photosensitizers of Chinese herbal medicines as HyPocrellin derivatives, psoralen, curcumin, hypericin, pseudohypericin, rheum emodin, riboflavin and aloe-emodin; and heptamethine cyanines as IR780 and IR775.

5. The preparation method according to claim 4, wherein, the photosensitizer is one or more of IR780, IR775 and phthalocyanin.

6. The preparation method according to claim 1, wherein, the emulsifier in Step (a) is one or more of lipids as DSPE-PEG2000, lecithin, cholesterol, DSPC, DPPC and DSPE; proteins as human albumin, hemoglobin, transferrin, immune globulin and insulin; macromolecules as PVA, poloxamer, Tween, Span, Brij, Myrj, polyoxyethylene and castor oil.

7. The preparation method according to claim 6, wherein, the emulsifier is one or more of phospholipid, DSPE-PEG2000 and albumin.

8. The preparation method according to claim 1, wherein, the protective agent for singlet oxygen in Step (b) is one or more of paraffin, lipiodol, soybean oil, dichloromethane, chloroform, perfluorinated compounds, deuteroxide and Freon 11.

9. The preparation method according to claim 1, wherein, the protective agent serves as the core material of microvesicle, micro capsule, particulate, micro emulsion, nanoparticle and nanoemulsion; or as the component of the film-forming material; or adheres to the film-forming material.

10. The preparation method according to wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent;
(b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.

11. The preparation method according to claim 1, wherein, the composition is used for improving the effect of photodynamic therapy.

12. The preparation method according to any of claims 2, wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent;
(b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.

13. The preparation method according to any of claims 3, wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent;
(b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.

14. The preparation method according to any of claims 4, wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent;
(b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.

15. The preparation method according to any of claims 5, wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent;
(b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.

16. The preparation method according to any of claims 6, wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent;
(b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.

17. The preparation method according to any of claims 7, wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent;
(b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.

18. The preparation method according to any of claims 8, wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent;
(b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.

19. The preparation method according to any of claims 9, wherein, the specific preparation method is as follows:
(a) Dissolving the photosensitizer and the emulsifier with a solvent under the conditions of 10-35° C. and pH 3-10 to obtain a mixed solution; using an amphiphilic lipid as the carrier carrying the hydrophobic photosensitizer and the emulsifier of the protective agent; dissolving the liposoluble photosensitizer and the amphiphilic emulsifier with the organic solvent; (b) Placing the mixed solution into an appropriate round-bottom flask and vacuumizing it, removing the solvent in a thermostat water bath so that the photosensitizer may evenly disperses into the hydrophobic end of the emulsifier; the emulsifier and the photosensitizer then form a uniform film at the bottom of the round-bottom flask;
(c) Adding the solvent into the round-bottom flask for ultrasonic hydration for 10-15 min so that the emulsifier can fall off and disperse into the water to form a micellar or vesicle structure, making the lipid film fall off from flask wall and evenly disperse into the solvent;
(d) Adding the protective agent into the solution obtained in Step (c) under the conditions of 2-35° C. and pH 3-9, dispersing it with a high-speed disperser; making the emulsifier wrap the protective agent through hydrophylic-hydrophobic interaction to form a nano or micron emulsion droplet.
